(19) United States
(12) Patent Application Publication
(10) Pub. No.: US 2024/0277719 A1
(43) Pub. Date: Aug. 22, 2024

(54) NOREPINEPHRINE REUPTAKE INHIBITORS FOR TREATING SLEEP APNEA

(71) Applicant: APNIMED, INC. (DELAWARE), Cambridge, MA (US)

(72) Inventors: Luigi TARANTO-MONTEMURRO, Brookline, MA (US); Ronald FARKAS, Clarksville, MD (US); David P. WHITE, Denver, CO (US); Lawrence G. MILLER, Cambridge, MA (US)

(21) Appl. No.: 18/570,673

(22) PCT Filed: Jun. 17, 2022
(86) PCT No.: PCT/US2022/033987

§ 371 Date: Dec. 15, 2023

Related U.S. Application Data
(60) Provisional application No. 63/211,673, filed on Jun. 17, 2021.
(60) Provisional application No. 63/319,035, filed on Mar. 11, 2022.

(51) Int. Cl. A61K 31/5375; A61K 9/00; A61K 31/216; A61K 31/496; A61K 31/5377; A61P 11/04

(52) U.S. Cl. CPC A61K 31/5375; A61K 9/0053; A61K 31/216; A61K 31/496; A61K 31/5377; A61P 11/04

(57) ABSTRACT

Methods of treating sleep apnea and snoring comprising administering a norepinephrine reuptake inhibitor, optionally in the absence of an antimuscarinic agent, and optionally as a monotherapy, are described herein. Pharmaceutical compositions comprising norepinephrine reuptake inhibitors, such as edivoxetine and viloxazine, are also described.

### CROSS-REFERENCE TO RELATED APPLICATIONS

This application claims the benefit under 35 U.S.C. § 119 (a) and (e) of and priority to U.S. Provisional Application No. 63/211,673, filed Jun. 17, 2021, and United U.S. Application No. 63/319,035, filed Mar. 11, 2022, the entire contents of each of which are incorporated herein by reference.

### TECHNICAL FIELD

The present invention provides methods of treating sleep apnea and snoring comprising administering a norepinephrine reuptake inhibitor, optionally in the absence of an antimuscarinic therapy, and optionally wherein the method of treatment is a monotherapy.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of a norepinephrine reuptake inhibitor (NRI).

Embodiments of this aspect of the invention may include one or more of the following optional features. In some embodiments, the method is performed in the absence of an antimuscarinic therapy. In some embodiments, the method excludes an antimuscarinic therapy. In some embodiments, the subject does not concurrently receive an antimuscarinic therapy, i.e., is not concurrently administered an antimuscarinic agent. In some embodiments, the NRI is reboxetine or a pharmaceutically acceptable salt thereof. In some embodiments, the NRI is edivoxetine or a pharmaceutically acceptable salt thereof. In some embodiments, the NRI is viloxazine or a pharmaceutically acceptable salt thereof. In some embodiments, the method is a monotherapy with reboxetine or a pharmaceutically acceptable salt thereof as the sole active pharmaceutical ingredient. In some embodiments, the reboxetine or pharmaceutically acceptable salt thereof is administered at a dosage of from about 1 mg to about 8 mg. In some embodiments, the reboxetine or pharmaceutically acceptable salt thereof is administered at a dosage of from about 2 mg to about 6 mg. In some embodiments, the reboxetine is administered daily. In some embodiments, the reboxetine or pharmaceutically acceptable salt thereof is (S,S)-reboxetine or a pharmaceutically acceptable salt thereof. In some embodiments, the NRI is atomoxetine or a pharmaceutically acceptable salt thereof. In some embodiments, the method is a monotherapy with edivoxetine or a pharmaceutically acceptable salt thereof as the sole active pharmaceutical ingredient. In some embodiments, the edivoxetine or pharmaceutically acceptable salt thereof is administered at a dosage of from about 5 mg to about 50 mg. In some embodiments, the edivoxetine or pharmaceutically acceptable salt thereof is administered at a dosage of from about 6 mg to about 36 mg. In some embodiments, the edivoxetine is administered daily. In some embodiments, the edivoxetine is administered in combination with oxybutynin, e.g., at a dose of from about 1 to about 20 mg of oxybutynin. The oxybutynin may be racemic oxybutynin or substantially enantiomerically pure R-oxybutynin. In some embodiments, the edivoxetine is administered in combination with trazodone or a pharmaceutically acceptable salt thereof, e.g., at a dose of from about 12.5 to about 200 mg. In some embodiments, the method is a monotherapy with viloxazine or a pharmaceutically acceptable salt thereof as the sole active pharmaceutical ingredient. In some embodiments, the viloxazine or pharmaceutically acceptable salt thereof is administered at a dosage of from about 50 mg to about 800 mg. In some embodiments, the viloxazine is administered daily. In some embodiments, the viloxazine is administered in combination with oxybutynin, e.g., at a dose of from about 1 to about 20 mg of oxybutynin. The oxybutynin may be racemic oxybutynin or substantially enantiomerically pure R-oxybutynin. In some embodiments, the viloxazine is administered in combination with trazodone or a pharmaceutically acceptable salt thereof, e.g., at a dose of from about 12.5 to about 200 mg. In some embodiments, the condition associated with pharyngeal airway collapse is sleep apnea, e.g., obstructive sleep apnea (OSA). In some embodiments, the condition associated with pharyngeal airway collapse is snoring, e.g., simple snoring. In some embodiments, the subject is in a non-fully conscious state. In some embodiments, the non-fully conscious state is sleep. In some embodiments, the NRI or pharmaceutically acceptable salt thereof is administered in an oral administration form. In some embodiments, the oral administration form is a syrup, pill, tablet, troche, capsule, or patch.

Another aspect of the invention provides a norepinephrine reuptake inhibitor for use in treating a condition associated with pharyngeal airway collapse.

Another aspect of the invention provides the use of a norepinephrine reuptake inhibitor in treating a condition associated with pharyngeal airway collapse.

Another aspect of the invention provides the use of a norepinephrine reuptake inhibitor for the manufacture of a medicament for treating a condition associated with pharyngeal airway collapse.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

FIGS. 2A and 2B are individual and group data showing the effect of placebo and reboxetine on AHI (4% definition for hypopneas) in 16 individuals with a previous diagnosis of OSA. Group data show medians and interquartile range. Placebo and reboxetine were administered approximately 1 week apart in random order for 1 night during a double-blinded crossover trial.

### DETAILED DESCRIPTION

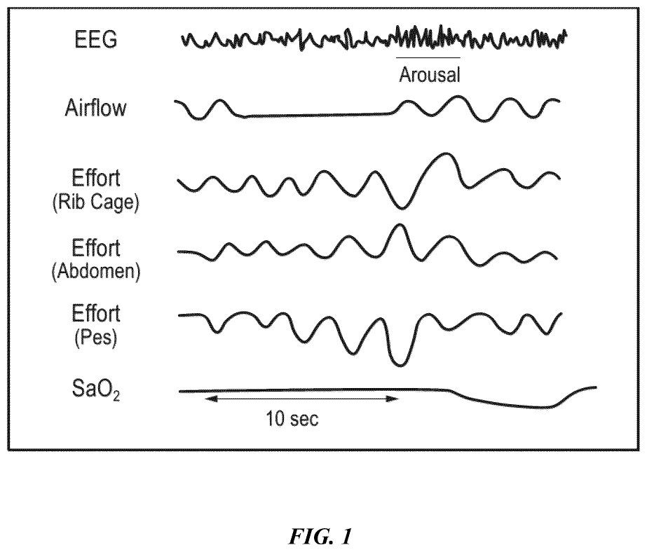

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009;32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI >5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 Aug; 100(2):416-21; Brownell et al., N Engl J Med 1982, 307:1037-1042; Sangal et al., Sleep Med. 2008 Jul;9(5):506-10. Epub 2007 Sep 27; Marshall et al. p. 2008 Jun;31(6):824-31; Eckert et al., Clin Sci (Lond). 2011 Jun; 120(12);505-14; Taranto-Montemurro et al., Sleep. 2017 Feb 1;40(2).

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, L. et al., The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019 May 15;199(10): 1267-1276.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of a norepinephrine reuptake inhibitor to a subject who is in need of, or who has been determined to be in need of, such treatment. In some embodiments, the NRI is reboxetine or a pharmaceutically acceptable salt thereof. In some embodiments, the NRI is edivoxetine or a pharmaceutically acceptable salt thereof. In some embodiments, the NRI is viloxazine or a pharmaceutically acceptable salt thereof.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, a “monotherapy” refers to the use of an agent individually (also referred to herein as alone), e.g., without another active ingredient to treat the same indication, e.g., sleep apnea or snoring. For example, in this context, the term monotherapy includes the use of reboxetine or a pharmaceutically acceptable salt thereof individually or alone to treat sleep apnea or snoring.

As used herein, an “antimuscarinic therapy” refers to the administration of an antimuscarinic agent. Antimuscarinic agents include but are not limited to atropine, propantheline, bethanechol, solifenacin, darifenacin, tolterodine, fesoterodine, trospium, oxybutynin, anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl. Subjects receiving treatment according to the present disclosure in the absence of an antimuscarinic therapy do not receive administration of an antimuscarinic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, Berge, S M. et al., “Pharmaceutical Salts,” J. Pharm. Sci., 1977;66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

In some embodiments, the NRI is a norepinephrine selective reuptake inhibitor (NSRI). In some embodiments, the NSRI is selected from the group consisting of amedalin, atomoxetine, 4-hydroxyatomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI). In some embodiments, the NNRI is selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, protryptyline, radafaxine, tapentadol, teniloxazine, and venlafaxine, or a pharmaceutically acceptable salt thereof. In some embodiments, the NRI is atomoxetine or a pharmaceutically acceptable salt thereof. In some embodiments, the NRI is 4-hydroxyatomoxetine or a pharmaceutically acceptable salt thereof. In some embodiments, the NRI is reboxetine or a pharmaceutically acceptable salt thereof. In some embodiments, the NRI is edivoxetine or a pharmaceutically acceptable salt thereof. In some embodiments, the NRI is viloxazine or a pharmaceutically acceptable salt thereof.

Reboxetine is the generic name of the pharmaceutical substance with the chemical name of 2-((2-ethoxyphenoxy)(phenyl)methyl)morpholine or 2-[α-(2-ethoxyphenoxy)benzyl]-morpholine, and its pharmaceutically acceptable salts. In various embodiments, reboxetine may be a racemic mixture of R,R- and S,S-enantiomers, or an isolated enantiomer, e.g., the S,S-enantiomer. In some embodiments, reboxetine may be reboxetine hydrochloride. In some embodiments, reboxetine may be reboxetine mesylate.

Edivoxetine is the generic name of the pharmaceutical substance with the chemical name of (1R)-2-(5-fluoro-2-methoxyphenyl)-1-[(2S)-morpholin-2-yl]-1-(oxan-4-yl)ethanol, and its pharmaceutically acceptable salts.

Viloxazine is the generic name of the pharmaceutical substance with the chemical name of 2-[(2-ethoxyphenoxy)methyl]morpholine, and its pharmaceutically acceptable salts.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

In some embodiments, the methods include administering a dose of from about 0.2 mg to about 12 mg of reboxetine or a pharmaceutically acceptable salt thereof. In some embodiments, the dose of reboxetine or a pharmaceutically acceptable salt thereof is from about 1 mg to about 8 mg. In some embodiments, the dose of reboxetine or pharmaceutically acceptable salt thereof is from about 0.5 mg to about 6 mg. In some embodiments, the dose of reboxetine or pharmaceutically acceptable salt thereof is from about 2 mg to about 6 mg. In some embodiments, the dose of reboxetine or pharmaceutically acceptable salt thereof is about 4 mg. In some embodiments, the dose of reboxetine or pharmaceutically acceptable salt thereof is about 6 mg. In some embodiments, the dose of reboxetine or pharmaceutically acceptable salt thereof is about 2 mg. In some embodiments, the dose of reboxetine or pharmaceutically acceptable salt thereof is about 3 mg.

In some embodiments, the reboxetine or pharmaceutically acceptable salt thereof is (S,S)-reboxetine or a pharmaceutically acceptable salt thereof. As used herein, (S,S)-reboxetine refers to the (S,S)-reboxetine stereoisomer substantially free of other stereoisomers of reboxetine.

In some embodiments, the reboxetine or pharmaceutically acceptable salt thereof is administered daily. In some embodiments, the reboxetine or pharmaceutically acceptable salt thereof is administered daily before sleep time, e.g., immediately before sleep time or 15-60 minutes before sleep time.

In some embodiments, the reboxetine or pharmaceutically acceptable salt thereof is administered in the absence of an antimuscarinic therapy.

In some embodiments, the reboxetine or pharmaceutically acceptable salt thereof is administered as a monotherapy.

In some embodiments, the reboxetine or pharmaceutically acceptable salt thereof is administered as a combination therapy with one or more additional active agents, i.e., which are not antimuscarinic agents.

In some embodiments, the methods include administering a dose of from about 5 to about 50 mg of edivoxetine or a pharmaceutically acceptable salt thereof. In some embodiments, the dose of edivoxetine or a pharmaceutically acceptable salt thereof is from about 6 mg to about 36 mg. In some embodiments, the dose of edivoxetine or a pharmaceutically acceptable salt thereof is from about 6 mg to about 20 mg. In some embodiments, the dose of edivoxetine or a pharmaceutically acceptable salt thereof is from about 20 mg to about 36 mg. In some embodiments, the dose of edivoxetine or a pharmaceutically acceptable salt thereof is from about 10 mg to about 25 mg.

In some embodiments, the edivoxetine or pharmaceutically acceptable salt thereof is administered daily. In some embodiments, the edivoxetine or pharmaceutically acceptable salt thereof is administered daily before sleep time, e.g., immediately before sleep time or 15-60 minutes before sleep time.

In some embodiments, the edivoxetine or pharmaceutically acceptable salt thereof is administered in the absence of an antimuscarinic therapy.

In some embodiments, the edivoxetine or pharmaceutically acceptable salt thereof is administered as a monotherapy.

In some embodiments, the edivoxetine or pharmaceutically acceptable salt thereof is administered as a combination therapy with one or more additional active agents.

In some embodiments, the edivoxetine or pharmaceutically acceptable salt thereof is administered as a combination therapy with oxybutynin or a pharmaceutically acceptable salt thereof (e.g., racemic oxybutynin or R-oxybutynin).

In some embodiments, the edivoxetine or pharmaceutically acceptable salt thereof is administered as a combination therapy with a hypnotic selected from the group consisting of trazodone, zolpidem, eszopiclone, benzodiazepines, gabapentin, tiagabine, and sodium oxybate, or a pharmaceutically acceptable salt thereof.

In some embodiments, the edivoxetine or pharmaceutically acceptable salt thereof is administered as a combination therapy with trazodone or a pharmaceutically acceptable salt thereof. In some embodiments, the trazodone or a pharmaceutically acceptable salt thereof is administered at a dose of from about 12.5 to about 200 mg. In some embodiments, the trazodone or a pharmaceutically acceptable salt thereof is administered at a dose of from about 12.5 to about 50 mg. In some embodiments, the trazodone or a pharmaceutically acceptable salt thereof is administered at a dose of from about 50 to about 200 mg. In some embodiments, the trazodone or a pharmaceutically acceptable salt thereof is administered at a dose of from about 25 to about 100 mg.

In some embodiments, the methods include administering a dose of from about 50 to about 800 mg of viloxazine or a pharmaceutically acceptable salt thereof. In some embodiments, the dose of viloxazine or a pharmaceutically acceptable salt thereof is from about 50 mg to about 300 mg. In some embodiments, the dose of viloxazine or pharmaceutically acceptable salt thereof is from about 300 mg to about 800 mg. In some embodiments, the dose of viloxazine or pharmaceutically acceptable salt thereof is from about 100 mg to about 500 mg.

In some embodiments, the viloxazine or pharmaceutically acceptable salt thereof is administered daily. In some embodiments, the viloxazine or pharmaceutically acceptable salt thereof is administered daily before sleep time, e.g., immediately before sleep time or 15-60 minutes before sleep time.

In some embodiments, the viloxazine or pharmaceutically acceptable salt thereof is administered in the absence of an antimuscarinic therapy.

In some embodiments, the viloxazine or pharmaceutically acceptable salt thereof is administered as a monotherapy.

In some embodiments, the viloxazine or pharmaceutically acceptable salt thereof is administered as a combination therapy with one or more additional active agents.

In some embodiments, the viloxazine or pharmaceutically acceptable salt thereof is administered as a combination therapy with oxybutynin (e.g., racemic oxybutynin or R-oxybutynin).

In some embodiments, the viloxazine or pharmaceutically acceptable salt thereof is administered as a combination therapy with a hypnotic selected from the group consisting of trazodone, zolpidem, eszopiclone, benzodiazepines, gabapentin, tiagabine, and sodium oxybate, or a pharmaceutically acceptable salt thereof.

In some embodiments, the viloxazine or pharmaceutically acceptable salt thereof is administered as a combination therapy with trazodone or a pharmaceutically acceptable salt thereof. In some embodiments, the trazodone or a pharmaceutically acceptable salt thereof is administered at a dose of from about 12.5 to about 200 mg. In some embodiments, the trazodone or a pharmaceutically acceptable salt thereof is administered at a dose of from about 12.5 to about 50 mg. In some embodiments, the trazodone or a pharmaceutically acceptable salt thereof is administered at a dose of from about 50 to about 200 mg. In some embodiments, the trazodone or a pharmaceutically acceptable salt thereof is administered at a dose of from about 25 to about 100 mg.

### Pharmaceutical Compositions

Also provided herein are pharmaceutical compositions comprising reboxetine, edivoxetine or viloxazine, or pharmaceutically acceptable salts thereof, as an active ingredient.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active pharmaceutical ingredients (APIs) for use in the present invention may be provided as pharmaceutically acceptable salts. In some embodiments, reboxetine is reboxetine hydrochloride. In some embodiments, reboxetine is reboxetine mesylate.

The APIs for use in the present invention may be formulated for immediate release or modified release, such as delayed release or extended release. For example, viloxazine or a pharmaceutically acceptable salt thereof may be formulated for immediate release (i.e., in an immediate release pharmaceutical composition) or for extended release (i.e., in an extended release pharmaceutical composition). Viloxazine is available in the United States as an extended release capsule.

For combination therapies described herein, the APIs may be formulated separately or together. In some embodiments, the APIs are formulated together, e.g., as a fixed dose combination. In some embodiments, the APIs are formulated separately, e.g., for concurrent administration.

Provided herein are pharmaceutical compositions comprising a pharmaceutically acceptable carrier or excipient and edivoxetine or a pharmaceutically acceptable salt thereof, and optionally further comprising oxybutynin or a pharmaceutically acceptable salt thereof.

Provided herein are pharmaceutical compositions comprising a pharmaceutically acceptable carrier or excipient and edivoxetine or a pharmaceutically acceptable salt thereof, and optionally further comprising trazodone or a pharmaceutically acceptable salt thereof.

Provided herein are pharmaceutical compositions comprising a pharmaceutically acceptable carrier or excipient and viloxazine or a pharmaceutically acceptable salt thereof, and optionally further comprising oxybutynin or a pharmaceutically acceptable salt thereof.

Provided herein are pharmaceutical compositions comprising a pharmaceutically acceptable carrier or excipient and viloxazine or a pharmaceutically acceptable salt thereof, and optionally further comprising trazodone or a pharmaceutically acceptable salt thereof.

In some embodiments, the dosages of edivoxetine, viloxazine, oxybutynin (e.g., racemic oxybutynin or R-oxybutynin), or trazodone, or pharmaceutically acceptable salts thereof, in a pharmaceutical composition may be a dose as described herein, e.g., for the treatment of sleep apnea or snoring.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 Apr; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

### EXAMPLES

The invention is further described in the following examples, which do not limit the scope of the invention described in the claims.

### Example 1. Reboxetine Crossover Study

A crossover study for treatment of OSA with reboxetine was performed. The study was a double-blind, randomized, placebo-controlled, cross-over, multi-centre study. On reboxetine night, participants received 4 mg of reboxetine mesylate. On placebo night, participants received matching placebo. Reboxetine and placebo nights were at least one week apart in random order. Participants received the reboxetine or placebo during visit for an acute, single night, laboratory sleep study. Participants had 2 overnight polysomonograms (PSGs). Oral administration of the treatment occurred immediately prior to bedtime.

The primary endpoint for the study was OSA severity measured by apnea/hypopnea index (AHI) representing the number of obstructions of the upper airway per hour sleep. Secondary endpoints were sleep parameters and phenotyping measures from PSG, sleep efficiency, next day sleepiness (Karolinksa Sleepiness Scale questionnaire) and next day alertness (driving simulator test).

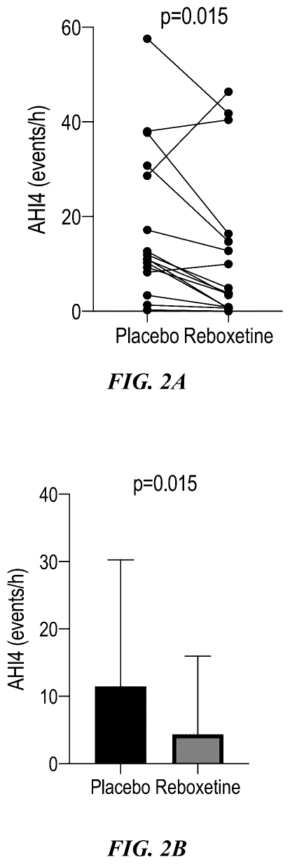

FIGS. 2A and 2B are individual and group data showing the effect of placebo and reboxetine on AHI (4% definition for hypopneas) in 16 participants from the crossover study with a previous diagnosis of OSA. Group data show medians and interquartile range.

### Example 2. Edivoxetine Crossover Study

A crossover study for treatment of OSA with edivoxetine is performed. The study is a double-blind, randomized, placebo-controlled, cross-over, multi-centre study. On edivoxetine night, participants receive edivoxetine. On placebo night, participants receive matching placebo. Edivoxetine and placebo nights are at least one week apart in random order. Participants receive the edivoxetine or placebo during visit for an acute, single night, laboratory sleep study. Participants have 2 overnight polysomonograms (PSGs). Oral administration of the treatment occurs immediately prior to bedtime.

The primary endpoint for the study is OSA severity measured by apnea/hypopnea index (AHI) representing the number of obstructions of the upper airway per hour sleep. Secondary endpoints are sleep parameters and phenotyping measures from PSG, sleep efficiency, next day sleepiness (Karolinksa Sleepiness Scale questionnaire) and next day alertness (driving simulator test).

### Example 3. Viloxazine Crossover Study

A crossover study for treatment of OSA with viloxazine is performed. The study is a double-blind, randomized, placebo-controlled, cross-over, multi-centre study. On viloxazine night, participants receive viloxazine. On placebo night, participants receive matching placebo. Viloxazine and placebo nights are at least one week apart in random order. Participants receive the viloxazine or placebo during visit for an acute, single night, laboratory sleep study. Participants have 2 overnight polysomonograms (PSGs). Oral administration of the treatment occurs immediately prior to bedtime.

The primary endpoint for the study is OSA severity measured by apnea/hypopnea index (AHI) representing the number of obstructions of the upper airway per hour sleep. Secondary endpoints are sleep parameters and phenotyping measures from PSG, sleep efficiency, next day sleepiness (Karolinksa Sleepiness Scale questionnaire) and next day alertness (driving simulator test).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of a norepinephrine reuptake inhibitor (NRI) in the absence of an antimuscarinic therapy.

2. The method of claim 1, wherein the NRI is administered as a monotherapy.

3. The method of claim 1 or 2, wherein the NRI is reboxetine or a pharmaceutically acceptable salt thereof.

4. The method of claim 3, wherein the reboxetine or pharmaceutically acceptable salt thereof is administered at a dosage of from about 1 mg to about 8 mg.

5. The method of claim 4, wherein the reboxetine or pharmaceutically acceptable salt thereof is administered at a dosage of from about 2 mg to about 6 mg.

6. The method of claim 1 or 2, wherein the NRI is edivoxetine or a pharmaceutically acceptable salt thereof.

7. The method of claim 6, wherein the edivoxetine or pharmaceutically acceptable salt thereof is administered at a dosage of from about 6 mg to about 36 mg.

8. The method of claim 1 or 2, wherein the NRI is viloxazine or a pharmaceutically acceptable salt thereof. 9 The method of claim 8, wherein the viloxazine or pharmaceutically acceptable salt thereof is administered at a dosage of from about 50 mg to about 800 mg.

10. The method of claim 1, wherein the NRI is edivoxetine or a pharmaceutically acceptable salt thereof, the method further comprising administering trazodone or a pharmaceutically acceptable salt thereof to the subject.

11. The method of claim 1, wherien the NRI is viloxazine or a pharmaceutically acceptable salt thereof, the method further rcomprising administering trazodone or a pharmaceutically acceptable salt thereof to the subject.

12. The method of any one of claims 1-11, wherein the NRI is administered daily.

13. The method of any one of claims 1-12, wherein the NRI is administered in an oral administration form.

14. The method of claim 13, wherein the oral administration form is a syrup, pill, tablet, troche, capsule, or patch.

15. The method of any one of claims 1-5, wherein the NRI is (S,S)-reboxetine or a pharmaceutically acceptable salt thereof.

16. The method of any one of claims 1-15, wherein the condition associated with pharyngeal airway collapse is sleep apnea.

17. The method of claim 16, wherein the condition associated with pharyngeal airway collapse is obstructive sleep apnea (OSA).

18. The method of any one of claims 1-15, wherein the condition associated with pharyngeal airway collapse is snoring.

19. The method of claim 18, wherein the condition associated with pharyngeal airway collapse is simple snoring.

20. The method of any one of claims 1-19, wherein the subject is in a non-fully conscious state.

21. The method of claim 20, wherein the non-fully conscious state is sleep.

22. A norepinephrine reuptake inhibitor for use in treating a subject having a condition associated with pharyngeal airway collapse in the absence of an antimuscarinic therapy, and optionally as a monotherapy.

23. A norepinephrine reuptake inhibitor for use in treating sleep apnea in the absence of an antimuscarinic therapy, and optionally as a monotherapy.

24. A norepinephrine reuptake inhibitor for use in treating snoring in the absence of an antimuscarinic therapy, and optionally as a monotherapy.

25. A method of treating a subject having a condition associated with pharyngeal airway collapse, the method comprising administering to a subject in need thereof an effective amount of (i) edivoxetine or a pharmaceutically acceptable salt thereof, and (ii) oxybutynin or a pharmaceutically acceptable salt thereof.

26. A method of treating a subject having a condition associated with pharyngeal airway collapse, the method comprising administering to a subject in need thereof an effective amount of (i) viloxazine or a pharmaceutically acceptable salt thereof, and (ii) oxybutynin or a pharmaceutically acceptable salt thereof.

27. The method of claim 25 or 26, wherein the oxybutynin is racemic oxybutynin or R-oxybutynin.

28. A pharmaceutical composition comprising (i) edivoxetine or a pharmaceutically acceptable salt thereof, (ii) oxybutynin or a pharmaceutically acceptable salt thereof, and (ii) a pharmaceutically acceptable excipient or carrier.

29. A pharmaceutical composition comprising (i) edivoxetine or a pharmaceutically acceptable salt thereof, (ii) trazodone or a pharmaceutically acceptable salt thereof, and (ii) a pharmaceutically acceptable excipient or carrier.

30. A pharmaceutical composition comprising (i) viloxazine or a pharmaceutically acceptable salt thereof, (ii) oxybutynin or a pharmaceutically acceptable salt thereof, and (ii) a pharmaceutically acceptable excipient or carrier.

31. A pharmaceutical composition comprising (i) viloxazine or a pharmaceutically acceptable salt thereof, (ii) trazodone or a pharmaceutically acceptable salt thereof, and (ii) a pharmaceutically acceptable excipient or carrier.

32. The pharmaceutical composition of claim 28 or 30, wherein the oxybutynin is racemic oxybutynin or R-oxybutynin.
